(19) United States
(12) Patent Application Publication
(10) Pub. No.: US 2010/0286053 A1
(43) Pub. Date: Nov. 11, 2010

(54) PLASMINOGEN ACTIVATOR INHIBITOR AMELIORATION OF NEWBORN HYPOXIC ISCHEMIC BRAIN INJURY

(75) Inventors: Chia-Yi Kuan, Loveland, OH (US); Daniel A. Lawrence, Ann Arbor, MI (US); Dian Er Yang, Cincinnati, OH (US); Faisal Adhami, Mason, OH (US)

Correspondence Address: THOMPSON HINE L.L.P.;Intellectual Property Group, P.O. BOX 8801, DAYTON, OH, 45401-8801, US

(73) Assignees: Children's Hospital Medical Center, Cincinnati, OH (US); The Regents Of The University of Michigan, Ann Arbor, MI (US)

(21) Appl. No.: 12/775,184

(22) Filed: May 6, 2010

Related U.S. Application Data
(60) Provisional application No. 61/176,401, filed on May 7, 2009.

(51) Int. Cl. A61K 38/57; C12Q 1/37; A61P 25/00

(52) U.S. Cl. 514/17.7; 435/23

(57) ABSTRACT

Plasminogen activators as potential therapeutic targets in neonatal hypoxia ischemia (HI) brain injury. Use of plasminogen activator inhibitor-1 (PAI-1) to ameliorate HI encephalopathy related disease. Use of PAI-1 as preventive treatment of cerebral palsy (CP).

This application claims priority from U.S. patent application Ser. No. 61/176,401 filed May 7, 2009 and incorporated by reference herein in its entirety.

This invention was made with government support under Grant No. R21NSO59668 awarded by the National Institutes of Health. The government has certain rights in the invention.

The disclosure encompasses plasminogen activators as potential therapeutic targets in neonatal hypoxia ischemia (HI) brain injury, uses of plasminogen activator inhibitors (PAI) to ameliorate HI encephalopathy related disease, and uses of PAI to prevent or ameliorate cerebral palsy (CP). One PAI is PAI-1. One PAI is neuroserpin. Amelioration includes improvement in motor, behavior, and/or cognitive function. PAI-1 may be wildtype or recombinant and may include mutations that do not significantly alter its activity as assessed by a person skilled in the art.

Disruption of the blood-brain-barrier (BBB) integrity is an important mechanism of neonatal HI brain injury. While tissue-type plasminogen activator (tPA) and matrix metalloproteinase-9 (MMP-9) can both produce BBB damage, their relationship in neonatal cerebral HI is unclear. The plasminogen activator (PA) system upstream of MMP-9 induction was evaluated in a rodent model and was required for the onset of neonatal HI brain injury. Cerebral ventricle injection of PAI-1 in rat pups after unilateral carotid artery occlusion followed by systemic hypoxia demonstrated that PAI-1 injection decreased the activity of both tPA and urokinase-type plasminogen activator (uPA) at 4 and 24 hrs of recovery, and blocked the HI-induced MMP-9 activation and BBB permeability at 24 hrs of recovery. Magnetic resonance imaging and histological assays indicated that the PAI-1 treatment regimen reduced brain edema, axonal degeneration, and cortical cell death at 24-48 hrs after HI. The PAI-1 treatment regimen showed a dose-dependent protection of brain damage at seven days of recovery, with the therapeutic window being at least four hrs after the HI insult. The brain PA system played a pivotal role in perinatal insult and may be a promising target to ameliorate effects of hypoxic-ischemic encephalopathy (HIE) in infants.

CP is a group of conditions that are characterized by chronic disorders of movement or posture. It is cerebral in origin, arises early in life, and is not the result of progressive disease. CP is also often accompanied by seizure disorders, sensory impairment, and cognitive limitation. Because CP is an umbrella clinical diagnosis, its etiologies are complex and diverse. Different CP subtypes show preferential prevalence in different gestational age groups. Spastic diplegia is the dominant form of CP in premature infants. Its basis, as indicated by neuroimaging and pathologic investigations, is often white-matter disorders. In congenital hemiplegic CP, the most common causes in term infants are perinatal stroke and/or congenital malformation. Quadriplegic CP, especially with dyskinesia, is often related to acute asphyxia during birth.

CP is particularly common among premature infants. The incidence of CP is 1.1 per 1000 live births in infants with >36 week gestational age, but rises to 40.4 in those of 28-31 weeks and 76.6 in premature infants <28 weeks. CP is linked to certain neurological conditions. Among them, periventricular leukomalacia (PVL, the odds ratio is 20.0), and germinal matrix hemorrhage with ventricular dilatation (PHVD, the odds ratio is 8.6) are the two most dangerous conditions. For comparison, the odds ratio of necrotizing enterocolitis (NEC) is 1.33.

There is no specific therapy for CP. Because germinal matrix hemorrhage and PVL are often considered part of spectrum presentation of neonatal hypoxic-ischemic encephalopathy (HIE), hypothermia therapy (head-cooling) may provide some benefit. However, hypothermia therapy was only given to term infants with HIE-like symptoms at present, and two large-scale clinical studies concluded that it has a lower efficacy than in animal models. Besides hypothermia therapy, the animal studies of neuroprotection in neonatal HIE were largely following cerebral ischemia studies in adults. But perhaps because none of the “animal-study-defined” neuroprotective agents for adult stroke in the past twenty years have shown substantial effects in clinical trials, no serious efforts were taken to translate animal studies of cytoprotective agents in neonatal HIE into clinical practice. There is a sizable disconnection between basic and clinical studies of neonatal HIE.

Recent studies indicate that neurovascular proteases, including matrix metalloproteases (MMPs) and secreted serine proteases, play a critical role in cerebrovascular diseases. This is because dysregulation of neurovascular proteases can degrade constituents of the extracellular matrix (ECM) and BBB, leading to brain edema, leukocyte infiltration, and the neuron-matrix detachment. In adult cerebral ischemia, MMP-9 has been suggested to be a promising therapeutic target, because its activity arises early in animal models and either genetic or pharmacological inhibition of MMP-9 offered brain protection (Heo et al., 1999; Asahi et al., 2001; Gu et al., 2005). In contrast, the role of MMP-9 in neonatal cerebral hypoxia-ischemia (HI) is unclear, because its activity was only detectable at 24 hrs of recovery, when much of irreversible brain damage has already occurred (Svedin et al., 2007). Thus, it seems unlikely that MMP-9 is a key initiator of neonatal HI brain injury.

tPA is another important neurovascular protease that may contribute to neonatal HI brain injury. tPA mainly circulates in the blood but also exists at low level in the brain parenchyma, which can be induced by excitotoxins to cause neurodegeneration. tPA also directly triggers the opening of BBB through activation of the latent platelet derived growth factor C, and elevates the MMP-9 levels after stroke. Cerebral HI produced rapid (<1 hr) and persistent tPA activation, up to 24 hrs, surrounding the blood vessels and lateral ventricles in newborn brains. The early-induction of the plasminogen activator (PA) system suggested that it may be an initial mechanism of neonatal HI brain injury.

Each cited reference is expressly incorporated herein in its entirety.

Intracerebroventricle (ICV) injection of PAI-1 was used to block the parenchymal tPA and urokinase PA (uPA) activity following the Vannucci model of cerebral HI in rat pups. Its effect on MMP-9 activation and brain damage were assessed. Results suggested that plasminogen activators are potential therapeutic targets in neonatal HI brain injury.

Seven-day-old Wistar rat pups were used for the cerebral ischemia-hypoxia model and ICV injection as previously described (Rice et al., 1981; Adhami et al., 2008). The percentage of tissue loss in the cerebral cortex, hippocampus, and striatum was quantified by comparison to individual counterparts on the contralateral hemisphere.

Plasminogen/casein zymogram, MMP zymogram, immunoblot, and immunocytochemistry were performed as previously described (Adhami et al., 2008). The procedure of quantifying Evans blue dye extravasation has been described (Su et al., 2008). A stable mutant form of human PAI-1 (Berkenpas et al., 1995) was purchased from Molecular Innovations (Southfield Mich.).

Magnetic resonance imaging data were acquired on a Bruker BioSpec 7T system with 40 G/cm gradients using a custom-built 25 mm single turn transmit/receive solenoid coil. Animals were scanned in two cohorts, with two each saline-treated or PAI-1-treated animals in the first cohort, and three each saline- or PAI-1-treated animals in the second cohort. Animals were brought to the scanner 24 hours after HI induction. They were anesthetized and maintained with 1% isoflurane in air and kept warm with heated air circulating through the magnet bore. T2-weighted anatomical images were acquired using a 2D RARE sequence with an effective echo time of 76.96 ms, repetition time of 1000 ms, field-of-view (FOV) of 19.2×19.2 mm², and a 256×192 matrix size. Diffusion tensor images were acquired with a spin echo sequence using an echo time of 21 ms, repetition time of 1100 ms, b-value of 800, 6 diffusion directions, FOV 19.2×19.2 mm², and a matrix of 128×128. For the second cohort, T2 maps were calculated from data acquired with a spin echo sequence using echo times of 20, 40, 60, 80, and 100 ms at a repetition time of 1800 ms with the same FOV and matrix as used for the diffusion scan.

Diffusion data were processed using Bruker's online processing software to calculate apparent diffusion coefficient (ADC) maps and directionally-color-encoded (DEC) maps of the fractional anisotropy (FA). T2 maps were calculated using Bruker's online data processing software.

Values are represented as mean±SD or SEM as indicated. Quantitative data were compared between different groups using Microsoft Excel's two-sample (unpaired) t-test assuming equal variance.

### BRIEF DESCRIPTION OF THE DRAWINGS

This application contains drawings executed in color. A Petition under 37 C.F.R. §1.84 requesting acceptance of the color drawings is filed separately on even date herewith.

FIGS. 1 A-D shows biochemical and BBB-permeability analysis at 4-24 hrs after neonatal cerebral hypoxia-ischemia.

FIGS. 2 A-I shows results of magnetic resonance imaging (MRI) at 24 hrs after hypoxia-ischemia.

FIGS. 3 A-O shows histological analysis at 48 hrs after hypoxia-ischemia.

FIG. 4 shows therapeutic efficacy of plasminogen activator inhibitor-1 (PAI-1) evaluated at 7 days after hypoxia-ischemia.

FIG. 5 is a schematic diagram of the role of plasminogen activator (PA) in neonatal HI brain injury.

FIG. 6 shows schemes for PAI-1 antagonized infection-sensitized HI brain injury and therapeutic PAI-1 administration.

FIG. 7 (A) shows multiple functional domains of tPA, (B) Generic mechanism of serpin-protease interactions, (C) PAI-1 disrupts vitronectin binding to the urokinase PA (uPA) receptor (uPAR) and α,β₃-integrins on migrating cells.

FIGS. 8 A, B, C shows tissue PA (tPA) production after cerebral HI.

FIGS. 9 A, B, C shows results of therapeutic PAI-1 administration.

FIGS. 10 A, B, C shows diffusion tensor imaging results.

FIGS. 11 A, B, C, D shows effects of PAI-1 on LPS and HI-induced blood brain barrier permeability.

FIGS. 12 A, B shows effects of PAI-1 administration on NFkB signaling and cytokine production.

FIGS. 13 A, B, C, D shows effects of PAI-1 administration on microglia activation.

FIGS. 14 A, B, C shows effects of PAI-1 administration on MMP and LPS and HI brain damage.

FIGS. 15 A, B, C shows effects of PAI-1 on motor development.

FIGS. 16 A, B, C, D shows neonatal HI injury on oligodendrocytes.

FIGS. 17 A, B, C, D, E shows effects of PAI-1 administration on motor development in cerebral palsy.

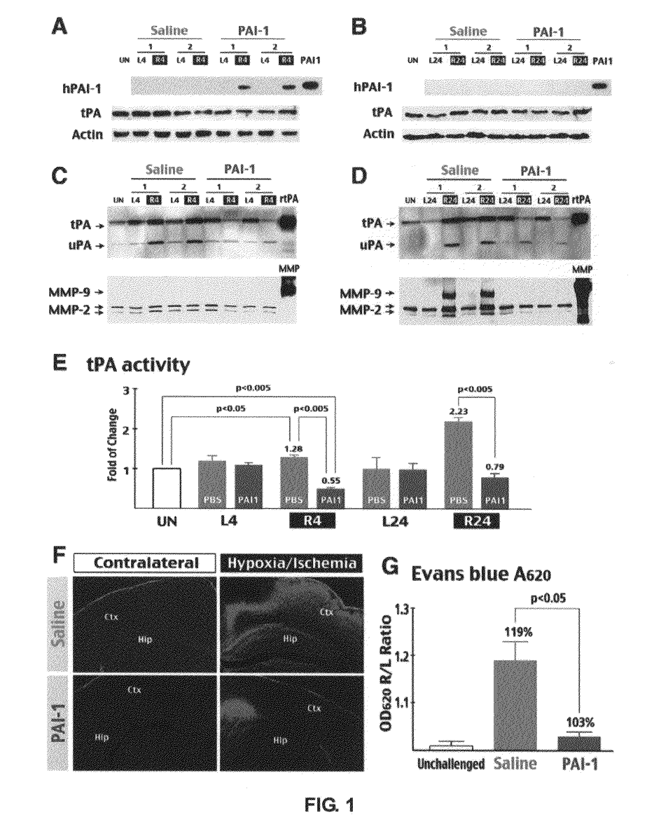

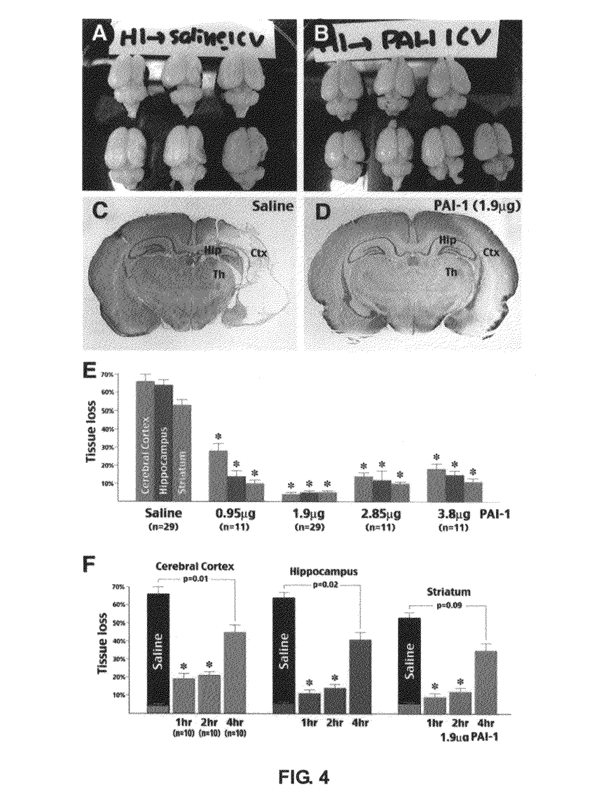

Biochemical and BBB-permeability analysis at 4-24 hrs after neonatal cerebral hypoxia-ischemia is shown in FIGS. 1A-D. Immunoblot analysis of recombinant PAI-1, tPA, and actin of brain samples at 4 (FIG. 1A) and 24 hrs (FIG. 4B) of recovery. R (right) is the HI-challenged side of brain, L (left) is contralateral side, and UN indicates unchallenged animals. Four representative sample sets are shown. of samples. Plasminogen/casein and gelatin zymogram gel analysis of brain samples at 4 (FIG. 1C) and 24 hrs (FIG. 1D). tPA and uPA activity were increased at both 4 and 24 hrs in saline-injected animals. The MMP-9 and MMP-2 activation was only detected at 24 hrs. PAI-1 injection dampened the induction of PAs and MMPs at both 4 and 24 hrs of recovery. Six representative sample sets are shown. Quantification of tPA activity as fold-change to the baseline level in unchallenged (UN) brain (FIG. 1E). A large area of immunoglobulin extravasation was detected on the hypoxia/ischemia-challenged side of brain in saline-injected animals, but not in PAI-1-injected animals or on the contralateral side (n=4) (FIG. 1F). Ctx: cerebral cortex; Hip: hippocampus. G, Quantification of Evans blue extravasation at 24 hr recovery (n=3). Shown in E & G are mean and standard error. p-value is determined by t-test.

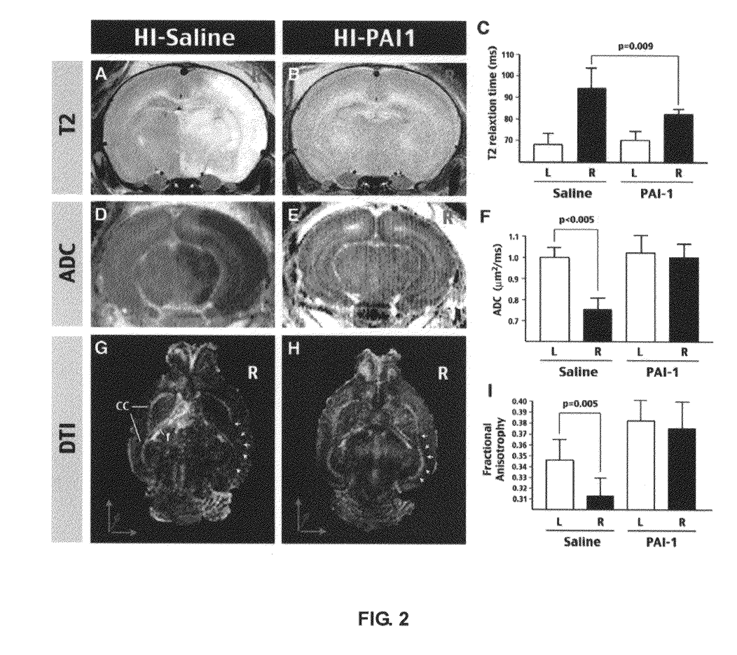

Magnetic resonance imaging (MRI) results at 24 hrs after hypoxia-ischemia are shown in FIGS. 2A-I. FIGS. 2A, 2B show T2-weighted image of HI-challenged and saline- (FIG. 2A) or PAI-1-treated (FIG. 2B) brain at 24 hrs. Saline-treated animals showed a large area of increased T2 signal intensity on the lesion (R) side. Quantification (FIG. 2C) showed T2 signal increased from 68 ms on the contralateral side to 95 ms on the ipsilateral side in saline-injected animals. By comparison, T2 signal increased from 72 ms on contralateral side to 83 ms on ipsilateral side in PAI-1-injected animals. The difference of increase from the contralaeral side was significant (p=0.009 by t-test, n=3). Diffusion-weighted image of saline-(FIG. 2D) or PAI-1-treated (FIG. 2E) brain at 24 hrs. There was a large area of decreased apparent diffusion coefficient (ADC) on the lesion side of saline-injected rat brains. Quantification (FIG. 2F) showed a significant reduction of ADC in saline-injected animals (p<0.005 by t-test, n=3). FIGS. 2G, H show diffusion tensor imaging (DTI) and quantitative directionally encoded color (DEC) map at 24 hrs. CC: corpus callosum; f: fibrim. There was absence of fibrim and posterior CC (arrows) in saline-injected (FIG. 2G), but not in PAI-1-injected (FIG. 2H) animal brain. Quantification (FIG. 2I) showed fractional anisotropy (FA) dropped from 0.347 on the contralateral side to 0.314 on the ipsilateral side in saline-injected rats (p=0.005 by t-test, n=5). There was no obvious difference of FA on contralateral side (0.383±0.018, mean ±SEM) and ipsilateral side (0.378±0.020) in PAI-1-treated animals (n=5).

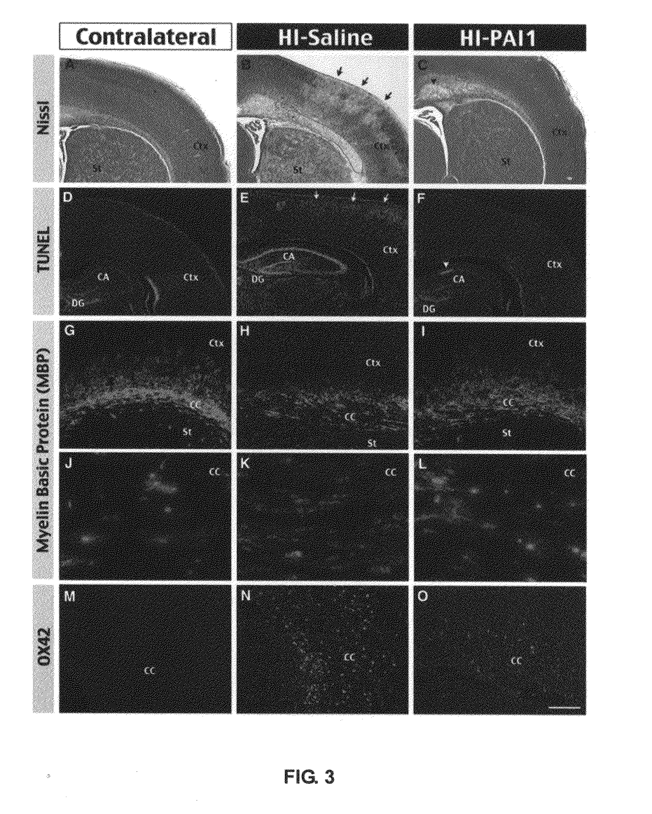

Histological analysis at 48 hrs after HI was performed using Nissl stain (FIGS. 3A-C), TUNEL stain (FIGS. 3D-F), anti-myelin basic protein (MBP) stain (FIGS. 3G-L), and anti-OX42 stain (FIGS. 3M-O) of the contralateral side (left column), HI-challenged and saline-injected (middle column) and HI/PAI-1-injected (right column) of rat brains at 48 hrs of recovery. Representative images in nine animals of each group are shown. There was expansion of corpus callosum (CC, delineated with red line) and columnar lesions (arrows) in saline-treated animal brains (FIG. 3B). Saline-treated brains also showed increased TUNEL-staining (FIG. 3E), fragmentation of MBP-positive processes (FIG. 3H) and reduced MBP-staining in the soma (FIG. 3K) of oligodendrocytes, and infiltration of OX42-positive macrophage in the CC (FIG. 3N). The PAI-1-treated brains showed localized CC swelling and TUNEL signals (arrowheads in FIGS. 3C, F). Ctx: cerebral cortex; St: striatum; CA: Ammon's horn of hippocampus; DG: dentate gyrus. Scale bar: 250 μm in FIGS. 3A-F; 100 μm in FIGS. 3G-I; 20 μm in FIGS. 3J-L; 50 μm in FIGS. 3M-O.

Therapeutic efficacy of PAI-1 was evaluated seven days after HI. Results are shown in FIG. 4. FIGS. 4A, B show examples of HI-challenged and saline-injected (FIG. 4A) or PAI-1-treated (FIG. 4B) rat brains at seven days of recovery. The majority of saline-injected animals showed significant tissue loss on the right side of the brain. FIGS. 4C, D are examples of Nissl-stained brain sections of saline-injected (C) or PAI-1-treated (D) rat brains. There was prominent cystic degeneration in the cerebral cortex of saline-treated brains. Ctx: cerebral cortex; Hip: hippocampus; Th: thalamus. FIG. 4E shows dose-response curve of tissue loss, compared to counterparts on the contralateral hemisphere, in the cerebral cortex (red), hippocampus (blue), and striatum (green) of animals receiving saline- or PAI-1 injection immediately after the HI insult. Asterisks: p<0.001 compared to saline-injected rats (n=11-29). FIG. 4F shows the therapeutic window of 1.9 μg PAI-1 injected at 1, 2, or 4 hrs after the cerebral HI insult. Asterisk: p<0.001 compared to the saline-injected animals. The p-values by t-test for 4-hr delayed injection compared to saline-injected animals were indicated.

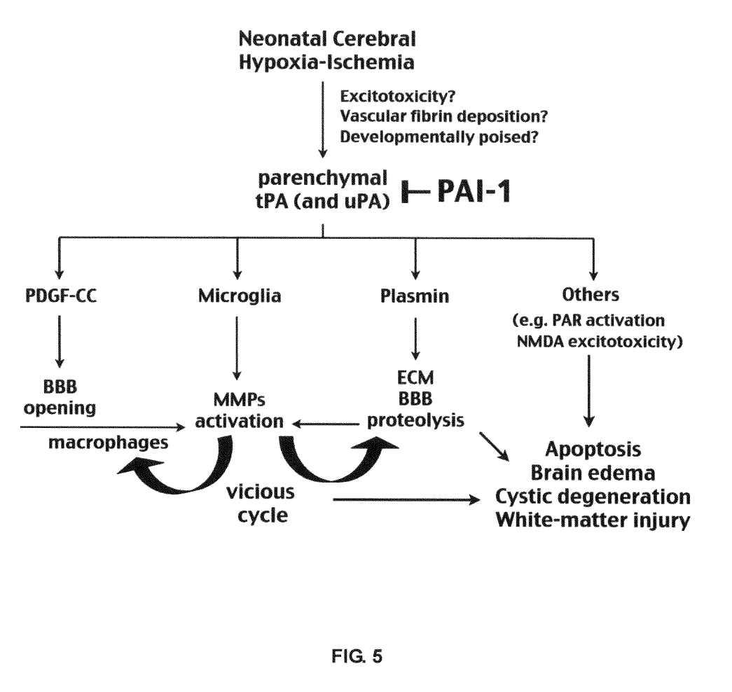

FIG. 5 is a schematic diagram indicating a pivotal role of PA in neonatal HI brain injury.

Inhibition of plasminogen activators blocked MMP-9 induction after HI. The relationship between plasminogen activators and MMP-9 in neonatal cerebral HI was evaluated by ICV-injection of saline or recombinant human PAI-1 (1.9 μg per pup) ipsilateral to the carotid artery occlusion at the end of a 90-minute hypoxic insult (10% O2) in P7 rat pups. Brains were collected at 4 or 24 hr recovery for biochemical analysis.

The immunoblot analysis showed that ICV-injected PAI-1 remained present on the HI-challenged (R) side of brain at 4 hrs of recovery, but was cleared at 24 hrs of recovery (FIGS. 1A, B; n=4). The HI insult or PAI-1 injection did not significantly alter the protein level of total tPA in the brain.

The plasminogen/casein zymogram showed a basal level of tPA activity in the brain, which was increased on the HI-stressed side at both 4 hrs (1.28-fold increase; p<0.05) and 24 hrs (2.23-fold; p<0.005; n=6) of recovery in saline-injected animals (FIGS. 1C-E). The tPA induction was accompanied by an increase of the uPA activity at both time-points in saline-injected animals. In contrast, the induction of MMP-9 and MMP-2 was only detectable at 24 hrs, but not 4 hrs, of recovery. The observed timings of PA and MMP-9 induction were consistent with those previously reported (Svedin et al., 2007; Adhami et al., 2008).

In contrast to saline-injection, the PAI-1 treatment greatly reduced the tPA activity at both 4 (0.55-fold of the basal level) and 24 hrs (0.79-fold of the basal level) of recovery (p<0.005 compared to saline-injection; n=6) (FIGS. 1C-E). The HI-induced uPA activity was also reduced to 57-78% of those in saline-injected animals. The PAI-1 injection completely blocked MMP-9 induction and reduced the MMP-2 activity (to 73% of saline-injection) at 24 hrs of recovery (FIG. 1D).

These results indicated that activation of the PA system preceded and was required for MMP-9 induction in neonatal cerebral HI.

PAI-1 therapy protected against HI-induced BBB permeability and brain edema. Because tPA and and MMP are both potent neurovascular proteases, the effect of PAI-1 therapy on HI-induced BBB permeability at 24 hrs of recovery was evaluated. Saline-injected animals consistently showed a larger area of immunoglobulin extravasation than PAI-1-injected animals on the HI-stressed side of brain (FIG. 1F, n=4). PAI-1 therapy also reduced the extent of Evans blue dye extravasation from 19% to 3% (p<0.05, n=3) (FIG. 1G). These results suggested more severe BBB damage in saline-injected animals.

A 7T magnetic resonance imaging (MRI) system further quantified the efficacy of PAI-1 therapy in preserving the BBB integrity after HI. Saline-injected, but not PAI-treated, animals exhibited a large area of increased T2 signal, which indicated water accumulation, on the ipsilateral hemisphere at 24 hrs of recovery (FIGS. 2A, B). A greater increase in T2 relaxation time over the contralateral side was found in saline-treated (68 ms to 95 ms; 37.5% increase) than PAI-1 treated animals (71 ms to 82 ms; 15.1% increase) (FIG. 2C, p<0.05 comparing the % increase from contralateral side; n=3).

The saline-injected animals also exhibited a large area of reduced apparent diffusion coefficient (ADC), a sign of diffusion-restricted intracellular space or tortuous extracellular pathway, which is generally used as an indicator of cytotoxic edema (Moseley et al., 1990), on the ipsilateral hemisphere (FIG. 2D). Quantification showed ADC dropped from 1.005±0.047 (μm²/ms) to 0.755±0.064 on the HI-challenged side in saline-injected animals (p<0.005; n=3) (FIG. 2F). In contrast, the reduction of ADC after HI was insignificant in PAI-1-animals (1.032±0.047 on contralateral side and 1.026±0.051 on ipsilateral side (FIGS. 2E, F; n=3).

These results indicated that inhibition of plasminogen activators reduced HI-induced BBB damage and brain edema in newborns.

PAI-1 therapy protected against HI-induced white-matter damage and cortical degeneration. Diffusion tensor imaging (DTI) evaluated if ICV-injection of PAI-1 protected against HI-induced white-matter injury, commonly used as an experimental model of periventricular leukomalacia (PVL) in infants (Volpe, 2008). Saline-injected rats showed approximately 10% reduction of fractional anisotropy (FA) in the corpus callosum (CC) at 24 hrs of recovery (p=0.005, n=5) (FIG. 2I). In contrast, there was little reduction of FA in the CC of PAI-1-treated animals (FIG. 2I). Quantitative directionally encoded color (DEC) map, a method to emphasize the orientation of anisotropic tissues (Chahboune et al., 2007), showed the absence of corpus callosum tract signal in saline-injected animals (arrows in FIG. 2G), but not in PAI-1-treated animals (FIG. 2H). These results suggest PAI-1 treatment ameliorated white-matter (WM) injury in neonatal cerebral HI.

Efficacy of PAI-1 therapy against HI-induced axonal damage was confirmed in saline- or PAI-1-treated animals at 48 hr recovery by histology (n=9 for each). All saline-injected animals showed brain damage, ranging from massive cystic degeneration to multiple columnar lesions associated with positive TUNEL-stain in the cerebral cortex (arrows in FIGS. 3B, E). The CC in saline-injected animals was typically swollen and de-fasciculated (outlined in FIG. 3B), whereas the PAI-1-treated animals exhibited isolated spots of CC swelling and TUNEL-stain (arrowhead in FIGS. 3C, F). No obvious lesion was found on the contralateral side of brain in saline- (FIG. 3A, D) or PAI-1-injected animals.

The saline-injected animals showed a greater decrease and fragmentation of myelin basic protein (MBP)-positive oligodendrocyte processes (FIG. 3H) and soma (FIG. 3K) in the CC, when compared to the contralateral side (FIGS. 3G, J) or PAI-1-injected animals (FIGS. 3I, L). The saline-injected animals tended to have more OX42-positive macrophages in the CC than the contralateral side or PAI-1-treated animals (FIGS. 3M-O). These results suggest that PAI-1 therapy reduced HI-induced white-matter injury and infiltration of inflammatory cells.

PAI-1 therapy protected against HI-induced brain damage. The therapeutic effect of ICV-injection of PAI-1 against cerebral HI at seven days of recovery was evaluated. The brains of saline- or PAI-1-treated animals in each experiment were photographed (FIGS. 4A, B), serial sectioned, and NissI-stained (FIGS. 4C, D). The extent of brain damage was quantified as the percentage of tissue loss in the cerebral cortex, hippocampus, and striatum compared to their counterparts on the contralateral hemisphere. For comparison, ICV-injection of α2-antiplasmin, an inhibitor of the downstream plasmin, but not PAs themselves, produced a maximal 55% reduction of tissue loss compared to contralateral counterparts if it was administered within two hrs after HI (Adhami et al., 2008).

For deriving the dose-response curve (FIG. 4E), saline or a varying dose of PAI-1 (0.95 to 3.8 μg) was injected within ten minutes after the HI insult. The extent of tissue loss in saline-injected animals was 67±3% (mean±standard error) in the cerebral cortex, 64±2% in the hippocampus, and 53±3% in the striatum (n=29). ICV-injection of PAI-1 in all doses significantly reduced tissue loss in all three regions (n=11-29 for each dose, p<0.001). 1.9 μg PAI-1 appeared to have the best protection, which decreased tissue loss to 4±1% in the cerebral cortex, and 5±1% in the hippocampus or striatum, when compared to contralateral counterparts (n=29).

To determine the therapeutic window (FIG. 4F), 1.9 μg PAI-1 was injected at 1, 2, or 4 hr recovery (n=10 for each), and the effects were compared to those following immediate post-HI injection. One- and two-hr delayed injection of PAI-1 still provided significant protection in all three regions when compared to saline-injection (p<0.001). Even four-hr delayed injection of PAI-1 reduced tissue loss to 45±4% in the cerebral cortex (p=0.01), 42±3% in the hippocampus (p=0.02), and 34±3% in the striatum (p=0.09). These results suggested that inhibition of PAs was a powerful strategy against neonatal cerebral HI injury.

PA is upstream of MMP in neonatal HI brain injury. It is known that uninhibited extracellular protease activity in the “neurovascular unit”, a conceptual entity that comprises neurons, microvessels that supply them, and the supporting cells, plays an important pathological role in cerebrovascular disorders (Mun-Bryce and Rosenberg, 1998; ladecola, 2004; Lo et al., 2004). These “neurovascular proteases” include MMPs, plasmin, plasminogen activators, and thrombin. Among them, MMP-9 and tPA have been discussed as potential therapeutic targets in adult ischemic stroke, because they both show early induction after injury, and either genetic or pharmacological inhibition of their activity offers protection in animal models (Asahi et al., 2001; Gu et al., 2005; Wang et al., 1998; Nagai et al., 1999; Yepes et al., 2000; Cinelli et al., 2001). There is little known about the interaction and relative roles between tPA and MMPs in neonatal cerebral HI.

The disclosed data revealed a causal relationship between PA and MMP in neonatal cerebral HI. It is known that HI induces rapid tPA and uPA activity (<4 hrs), whereas the MMP-9 induction occurs late (at 24 hrs) in the newborn brain (Svedin et al., 2007; Adhami et al., 2008). Early PA inhibition after neonatal HI insult was sufficient to prevent subsequent MMP activation, suggesting that PA is upstream of MMP.

PA plays a pivotal role in neonatal HI brain injury. ICV injection of PAI-1 provided a greater degree of protection and a longer therapeutic window against neonatal HI brain injury than targeting plasmin or MMP-9 activity, as shown in previous studies (Svedin et al., 2007; Adhami et al., 2008). This pattern of differential efficacy suggested a model in which PA, or tPA alone, plays a key initiator role in neonatal HI brain injury (FIG. 5). Specifically, the HI-induced tPA may directly trigger the opening of BBB allowing blood-borne cells to enter the brain parenchyma, leading to an increased inflammatory response and MMP activity (Su et al., 2008; McColl et al., 2008). tPA can also function as a cytokine to stimulate microglia to secrete more MMPs (Siao and Tsirka, 2002). The tPA-converted plasmin is a broad-spectrum serine protease that can degrade many constituents of the ECM and BBB, which may in turn amplify MMP induction as a secondary response (Tsirka et al., 1995). Thus, MMP induction is part of consequences of tPA toxicity in neonatal HI brain injury. However, because MMPs are potent collagenases that can cause severe damage to the vascular wall, their activation may accelerate a transformation from transient to sustained BBB disruption (del Zoppo et al., 2007; McColl et al., 2008).

These results suggested that inhibition of the upstream PA activity was a more effective strategy of brain protection in neonatal HI than targeting any of the downstream effectors. Although the toxicity of the PA system is mostly attributed to tPA, the roles of tPA and uPA in this pathological process warrant comparison.

PA was a potential therapeutic target in neonatal HIE. The experimental (Vannucci) paradigm used is a popular model of neonatal HIE (Rice et al., 1981). HIE is an important cause of perinatal mortality and long-term neurological morbidity (e.g. cerebral palsy and mental retardation), but the current therapy of HIE is mainly supportive (Ferriero, 2004; Volpe, 2008). Hence, developing effective strategies of brain protection against HIE remains an urgent issue in neonatology. The disclosed results have clinical implications because inhibition of PAs prevented oligodendrocyte injury (similar to periventricular leukomalacia) and widespread tissue degeneration (similar to cystic encephalomalacia) in the rodent model of HIE, and provide a brain protection strategy in HIE.

Infants diagnosed with HIE or at a high risk of CP may have greater levels of tPA and plasmin in the brain or the cerebrospinal fluid. Inhibition of the extravascular PA system may ameliorate this devastating perinatal disorder.

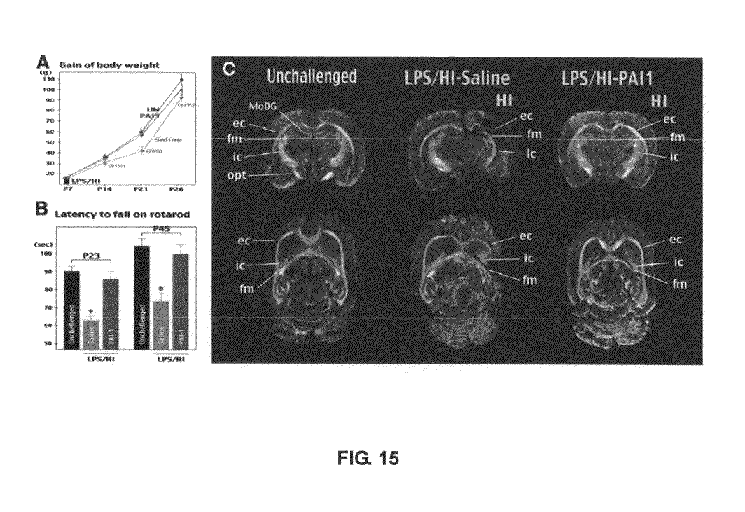

Efficacy and dose-determination of PAI-1 therapy in an inflammation (LPS)-sensitized neonatal cerebral hypoxia-ischemia model in rats. Histopathology at seven days after HI is evaluated. Motor-behavior and learning ability assay at two months is evaluated. Results are shown in FIG. 15. PAI-1 administration at P7 protected motor coordination function on rotarod tests in infection-HI-challenged rat pups when they reached two months of age. MRI-DTI examination showed PAI-1 administration preserved WM integrity in infection/HI-challenged rat pups to a degree that was almost identical to that of unchallenged rats of the same age.

Pharmacokinetic, efficacy and safety analysis of the GMP grade PAI-1 in rats and rabbits is expected to show no toxicity or thrombosis.

Hypoxia-ischemia and intrauterine infection are two principal mechanisms of hypoxic-ischemic encephalopathy (HIE) and periventricular leukomalacia (PVL), which often lead to cerebral palsy (CP). There incidence of HIE and PVL is particularly higher in premature infants, but the best therapeutic option, hypothermia, is not applicable to premature infants and is less effective in term infants with severe HIE. More effective therapies of HIE and PVL remain a critical issue in neonatal care. Infection alone may cause the disclosed pathological effects. Infection may be a co-morbidity with other conditions that sensitizes the extent of HI damage.

Tissue-type plasminogen activator (tPA) promotes fibrinolysis in the blood, but a high level of tPA activity in the brain parenchyma has many harmful effects. Immature brains have a unique response to hypoxia-ischemia (HI) by a persistent induction of plasminogen activators (PA), leading to severe tissue proteolysis in the parenchyma. Intracerebroventricular (ICV) injection of plasminogen activator inhibitor-1 (PAI-1) significantly reduced the HI-induced brain injury in rat pups. PAI-1 administration was protective against lipopolysaccharide (LPS)-sensitized HI brain injury in newborns, suggesting therapy against infection-sensitized HIE/PVL. While infection causes a greater risk of CP and PVL, there are few effective therapies against infection-sensitized HIE/PVL.

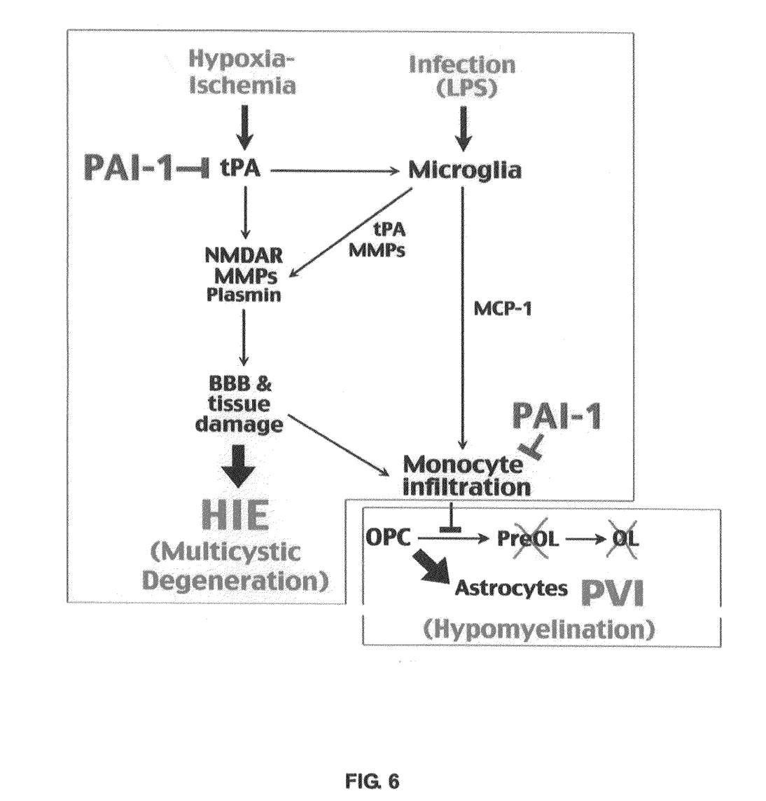

FIG. 6 The mechanism by which PAI-1 protected against perinatal infection/HI brain injury, and the efficacy of the experimental therapy for HIE and PVL, was evaluated. PAI-1 antagonizes infection-sensitized HI brain injury by two main mechanisms: neutralizing tPA activity, and blocking vitronectin-mediated recruitment of monocytes into the brain, presumably by monocyte chemoattractant protein-1 (MCP-1). Hypomyelination in PVL is caused by infection/HI-induced death as well as aberrant differentiation of oligodendrocyte precursors (OPCs). PAI-1 administration mitigates/ameliorates deleterious effects. Postnatal PAI-1 administration protects long-term WM development against LPS-/HI injury.

To determine the mechanisms by which PAI-1 antagonizes the LPS/HI brain injury, the effects of ICV-administration of stable PAI-1, a stable PAI-1 mutant without vitronectin-binding affinity, and α2-antiplasmin against LPS-sensitized HI injury were compared in rat pups. A greater efficacy with stable PAI-1 was predicted, due to its dual-action of blocking tPA activity and vitronectin-mediated monocyte transmigration across blood-brain-barrier.

The ability of PAI-1 and the vitronectin non-binding PAI-1 mutant was compared in blocking the recruitment of pre-labeled monocytes after LPS/HI brain injury or ICV-injection of various combinations of LPS, tPA, and MCP-1 in wild-type or MCP-1-null mouse pups. Key chemoattractants for monocyte recruitment in LPS-sensitized HI brain injury and the critical functional domain of PAI-1 for blocking this process may be shown.

To test if PAI-1 protects oligodendrocyte progenitor (OPC) survival and differentiation, a genetic method (NG2-Cre mice carrying a stop-floxed EGFP marker) was used to fate-map the progeny of NG2+progenitors, which normally only produce oligodendrocytes in the dorsal cortex, following LPS/HI brain injury and PAI-1 or saline therapy. The presence of EGFP+ (hence, the progeny of NG2+progenitors) astrocytes in the corpus callosum and dorsal cortex due to aberrant OPC differentiation was predicted.

Time-lapse videomicroscopy in brain slices was used, as in a previous study, to visualize OPCs following LPS/HI insults and PAI-1-versus-saline therapy.

The ultimate benchmark for success in therapy of HIE and PVL is maintenance of normal WM development. To test if PAI-1 therapy protected WM development against pre/post-natal LPS/HI injury, the Diffusion Tensor Imaging (DTI) methodology was developed to evaluate the integrity of WM in rodents.

DTI and stereology-based counting of oligodendrocytes in CNP-EGFP mice were used to determine the ability of PAI-1 in preserving WM development after neonatal LPS/HI injury, and to test if postnatal PAI-1 therapy was a useful add-on or primary therapy for prenatal infection/LPS-induced WM injury. Positive outcomes support the potential of PAI-1 administration as a new therapy for perinatal HIE and PVL injury.

Infection worsens HIE and PVL. Several lines of evidence indicated that intrauterine infection and the fetal inflammatory response either directly leads to WM injury or increases brain vulnerability to secondary perinatal insults such as HI. For example, epidemiological studies indicated that chorioamnionitis is a high risk factor for CP and PVL especially among premature infants. Exposure of fetuses to the bacterial endotoxin LPS led to WM injury in the pups. It is known that administration of LPS at 4-6 hr before HI also significantly increased brain injury in P7 rats. Because premature infants are excluded from the hypothermia therapy, there is an urgent need to develop treatments of infection-HI injury in this high-risk population.

In animal models, MyD88-null mice showed the same degree of brain damage in neonatal HI, but they are resistant to LPS-sensitization, suggesting a critical involvement of the TLR-mediated innate immunity in this response. In pharmacological studies, while many drugs are known to decrease infection/LPS- or HI-induced neonatal brain injury, few reagents can protect against the combination of both. The best studied neuroprotectant for LPS-sensitized HI injury in animal model is N-acetylcysteine (NAC), a free radical scavenger. Injection of multiple doses of NAC, both before and after the HI insult, reduced brain injury in rat pups, but the efficacy of post-LPS/HI administration of NAC is unknown. Therefore, developing additional therapies against LPS-sensitized perinatal HI brain injury remains an important goal for better neonatal care.

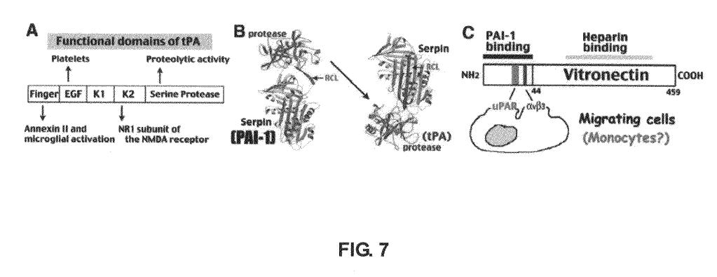

The toxicity of tPA. tPA is a highly specific serine protease and a principal plasminogen activator (PA) in blood for the fibrinolytic activity. Although tPA is currently the only FDA-approved agent for thrombolytic therapy in ischemic stroke, it also has many deleterious effects outside the blood vessels. It is known that excitotoxins induces tPA synthesis by microglia and astrocytes, leading to neurodegeneration. The multitude of detrimental effects of tPA are mediated by its many functional domains besides the serine protease activity (FIG. 7A). For example, unlike uPA, tPA has the second kringle domain (K2) to bind to the NMDA receptor NR1 subunit, leading to its cleavage and enhanced glutamate excitotoxicity. tPA stimulates microglia activation using the Finger domain to bind to Annex II, and it has been implicated in the activation of NF-kB signaling in microglia in stroke. tPA induces matrix metalloproteinase (MMPs) in the neurovascular unit, resulting in hemorrhagic transformation following ischemic stroke. tPA directly induces the opening of BBB via a LRP- and PDGF-CC-mediated signaling pathway. These studies suggest that excessive tPA activities in the brain parenchyma may produce many detrimental effects beyond plasminogen conversion. This consideration explains why only tPA, but not the other plasminogen activator (uPA), is associated with neurotoxicity in the brain.

While thrombolytic therapy with exogenous tPA has benefit, the roles of endogenous tPA in adult ischemic stroke are controversial. A smaller infarct volume in tPA-null mice has been reported in one paradigm, while others showed opposite effects with another model. One potential explanation to the conflicting reports is that some experimental models may activate the brain parenchymal tPA stronger than the others. Moreover, the age of animals is a critical determinant. For example, we have shown that unilateral carotid occlusion plus hypoxia (the Levine/Vannucci model) did not activate PAs in adult rodent brains. Yet, the same insult triggers fast and persistent tPA and uPA activity, leading to extensive proteolysis-type damage to immature brains, which is largely preventable with ICV-injection of PAI-1. Preliminary results indicated that PAI-1 administration markedly decreased the LPS-sensitized HI brain injury in neonatal rats. These results suggest that the parenchymal tPA is a promising therapeutic target in perinatal HI brain injury.

Multiple biological functions of PAI-1. PAI-1, a 50 kD single-chain glycoprotein, belongs to the serine protease inhibitor (serpin) family. FIG. 7A shows multiple functional domains of tPA. FIG. 7B shows a generic mechanism of serpin-protease interactions. FIG. 7C shows that PAI-1 disrupts vitronectin-binding to the uPA receptor uPAR) and ᵥβ₃-integrins on migrating cells.

The molecular mechanism by which serpin inhibits target-protease has been determined (FIG. 7B). During this process, serpin uses the reactive center loop (RCL) as a “bait” to tangle with its target, leading to the cleavage of RCL, which triggers a large conformational change in the serpin that involves rapid insertion of the RCL to the four R-strands in the main structural feature of serpin. This results in tight locking of a serpin-protease complex, as well as deformation-inhibition of the target protease. PAI-1 inhibits tPA and uPA with a second-order rate constant (˜10⁻⁷ M⁻¹S⁻¹) 10-1000 times faster than other serpins. PAI-1 also directly inhibits plasmin and is the key regulator of plasminogen conversion in the blood. Because PAI-1 in the brain is scarce and unstable, it does not form a physiological defense mechanism against tPA neurotoxicity in the CNS.

Besides serine proteases, PAI-1 interacts with many other substrates that have important, but less characterized functions, e.g., PAI-1 interacts with the lipoprotein receptor-related protein (LRP) to accelerate the clearance of LRP-bound tPA and uPA/uPA receptor (uPAR) complex. PAI-1 also binds with high affinity to vitronectin (VN), a major adhesion molecule in the extracellular matrix. The uPAR and integrin αᵥβ₃ receptor, often on the leading edge of migrating cells, bind to the same domain in VN but with a lower affinity than PAI-1 (FIG. 7C). Hence, PAI-1 inhibits smooth muscle cell migration on the vitronectin substrate. Up-regulation of the cell surface uPAR expression promotes monocyte infiltration in acute myocardial infarction and, if a similar scenario occurred in neonatal cerebral HI, PAI-1 administration could limit monocyte brain infiltration.

Two PAI-1 mutants are of particular interest: a stable PAI-1 (CPAI) and a vitronectin non-binding mutant (PAI1-AK). The CPAI mutant has 4 amino acid substitutions (N150H, K154T, Q319L, M354I) which extends the half-life of PAI-1 from 2 to 145 hours. The CPAI mutant has been used to protect newborn rat brains from HI injury. PAI1-AK has the same mutations of CPAI and 2 additional amino-acid substitutions (R101A/Q123K), resulting in no detectable vitronectin-binding activity but intact protease inhibition. These two PAI-1 mutants are used to determine the importance of VN-binding activity for PAI1-mediated protection in neonatal LPS/HI brain injury.

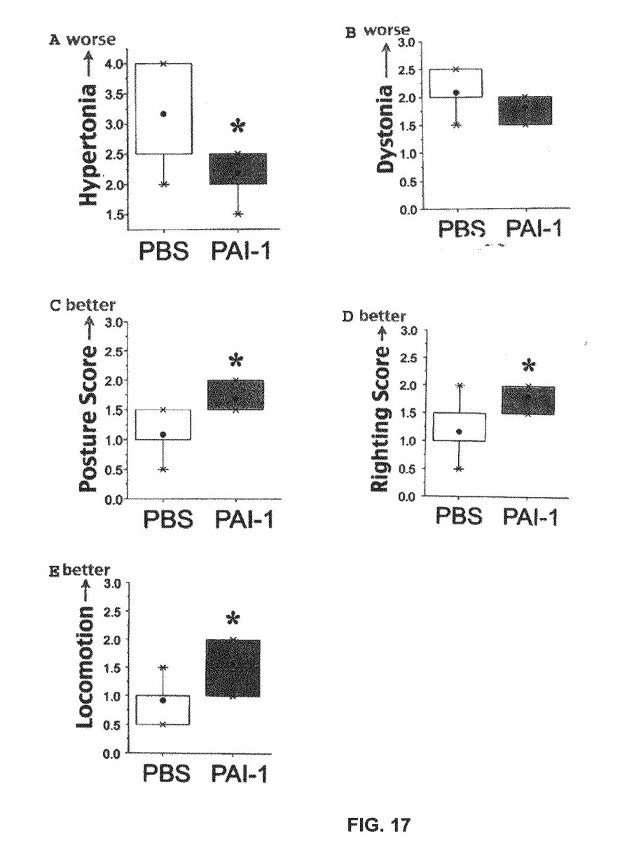

The mutant PAI-1 containing 4 amino acid substitutions was used. This mutation extended the half-life of PAI-1 without altering its functions or specificity. Results are shown in FIGS. 17 A, B, C, D, and E. PAI-1 administration significantly decreased prenatal HI-induced CP-like symptoms including hypertonia and dystonia in rabbits. The motor functions, such as posture score, righting reflex, and locomotion activities were also improved by PAI-1 administration.

PAI-1 inhibitors ameliorated behavioral deficits caused by near-term HI. The effect of HI at different gestational ages revealed that near-term fetuses suffered more mortality with a similar extent of HI compared to preterm gestation. Using MRI, apparent diffusion coefficients (ADC) started dropping after a few minutes, and dropped below a threshold level only if the fetus was destined to become hypertonic at a preterm gestation. The same level of ADC-drop was reached at 30 min of HI at embryonic day (E) 29 as 40 min of HI at E25. A cohort of E29 rabbits were subjected to 30-32 min of HI and delivered at E30. Those rabbit kits were administered either PAI-1 or an equal volume of PBS into the subarachnoid space via the posterior fontanelle. The position of the 26 gauge needle in the subarachnoid space was verified in a group of kits before. Rabbit kits were then put in an incubator, monitored closely, and fed by oro-gastic route. Two days later, at E32 (P1) a battery of neurobehavioral tests were conducted, including the forced swim test. The HI affected kits receiving PAI-1 injection (n=8) had significantly better behavior outcomes, compared to those receiving PBS (n=6). Improvements in posture, locomotion, tone, and righting reflex were observed (p<0.05, t-test) and the dystonia score showed a trend of improvement.

Impaired oligodendrocyte development underlies PVL. Hypomyelination is a pathological hallmark of PVL, indicating that impaired oligodendrocyte development is a key cellular basis of this condition. Oligodendrocyte progenitors (OPCs) and immature oligodendrocytes (OLs) are particularly sensitive to HI- or LPS- induced insults, in part due to a specific up-regulation of edited GIuR2-free, Ca²⁺-permeable AMPA receptor (thus behaving like the NMDA receptor). Consistent with this hypothesis, local injection of ibotenic acid, a NMDA receptor agonist, induced OPC degeneration and neonatal WM injury, but tPA-null mice have a higher resistance to this insult, suggesting that tPA enhances the excitotoxicity-induced perinatal WM injury.

Besides OPC death, there is a robust regenerative response after HI, but the replenishing OLs are stuck in an “arrested maturation” state in chronic perinatal WM injury). There may be aberrant differentiation by the surviving OPCs or OLs, because NG2+ OPCs are capable of generating both oligodendrocytes and astrocytes. However, the current data are entirely based on immunostaining of OL differentiation markers, such as O4 and O1, which do not allow direct visualization of OPCs or reveal morphological details of the progeny. Genetic fate-mapping strategy (using NG2-Cre mice carrying a Cre-dependent an EGFP tracer) are used to examine how LPS/HI impairs, and whether PAI-1 rescues, the OPC survival and differentiation.

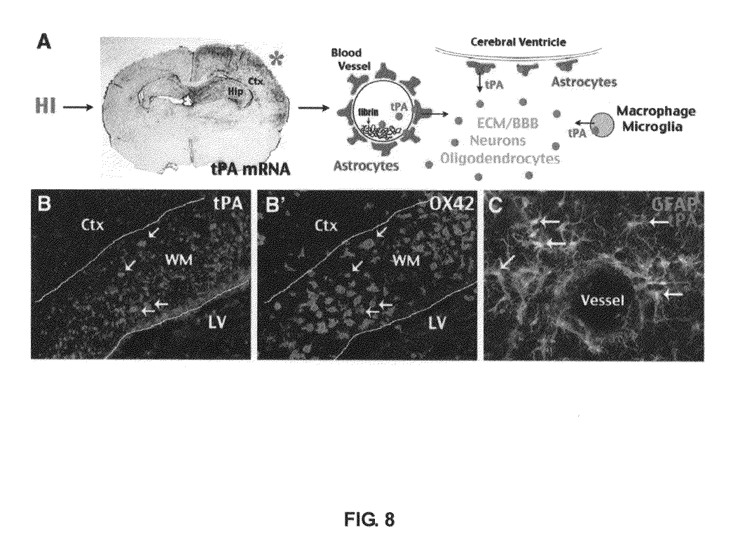

tPA-producing cells in neonatal cerebral HI were identified using in-situ hybridization against tPA mRNA, and immunocytochemistry double-labeling (FIG. 8). Astrocytes and microglia produce tPA after cerebral HI in newborn animals. in-situ hybridization showed tPA mRNA induction on the HI-stressed side of brain (FIG. 8A asterisk). Double-labeling demonstrated that astrocytes and microglia synthesized tPA. FIG. 8B, B′ showed double-labeling of tPA mRNA and activated microglia=macrophage marker OX42 . FIG. 8C showed merged tPA/GFAP image; the arrows denote co-localization. HI induced tPA expression on the prospective lesion-side of brain as early as 4 hrs after the insult (FIG. 8A). tPA was produced by OX42+ microglia/macrophages in WM and by GFAP+ astrocytes outside blood vessels (FIGS. 8B, C). This pattern of extravascular tPA expression raised the possibility of neurotoxicity.

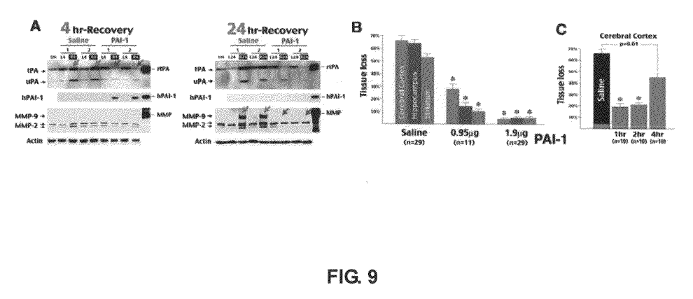

The efficacy of α2-antiplasmin, PAI-1, and neuroserpin against HI were compared to deduce the key mediator(s) of neonatal HI brain injury. FIG. 9A shows early PA-inhibition prevented the HI-induced, late-onset of MMP activity. FIG. 8B shows results of a dose-response study; a therapeutic regimen of PAI-1 significantly decreased brain tissue loss at seven days after HI. FIG. 9C shows results of a therapeutic window study; ICV-injection of PAI-1 at four hrs after HI remained neuroprotective. The following agents affect different targets in the plasminogen system: α2-antiplasmin inhibits plasmin; PAI-1 inhibits both tPA and uPA; neuroserpin preferentially inhibits tPA. PAI-1 mitigated 90% of brain tissue loss and prevented HI-induced MMP activation. The maximal effect of antiplasmin was 50%-reduction of tissue loss (FIG. 9). These results indicated that plasminogen activators (PAs), but not plasmin, were the key mediators of HI brain injury in newborns.

The efficacy of neuroserpin was evaluated. Neuroserpin protected against HI brain injury as potently as PAI-1, it was much less effective against LPS-sensitized HI (FIG. 9 and data not shown). Unlike PAI-1, neuroserpin does not have any VN-binding activity. This disparity suggested the possibility that while tPA-inhibition was sufficient to block neonatal HI brain injury, the additional PAI-1-specific action of interference with VN-mediated monocyte transmigration was needed to resist the infection/LPS-sensitized HI insult.

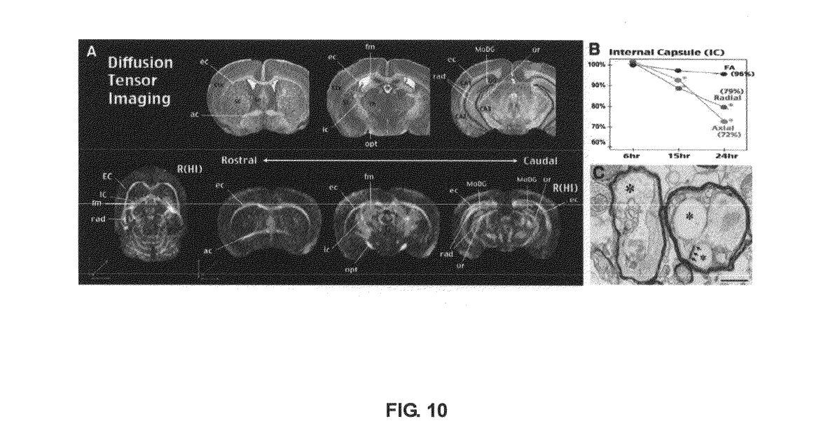

Diffusion tensor imaging (DTI) captured HI= induced acute axonal injury. FIG. 10A shows color-coded DTI images of an adult mouse at 15 hrs after a HI-insult on the right (R) side of the brain. Quantification (FIG. 10B) shows DTI parameters in the internal capsule (ic). EM examination showed swelling of myelin sheaths and compression of the axoplasma (FIG. 10C) which can account for reduced radial diffusivity. MRI-DTI is used to evaluate perinatal WM injury in clinical practice due to its superior ability to provide three-dimensional tractography and quantitative measurements of directional water diffusion in axonal tracts. In studying animal models of perinatal HI brain injury, it is preferable to use the same method as in clinical settings to assess long-term therapy effects. Both in-vivo and ex-vivo DTI methodology was used to evaluate the integrity of WM in adult mice (FIG. 10) and rat pups. The DTI method detected all major axonal tracts in the forebrain of adult mice, and showed a progressive reduction of axial/longitudinal as well as radial/transverse water diffusivity in the HI-challenged internal capsule (FIGS. 10A, 10B). EM studies of the DTI-imaged sample demonstrated the swelling of myelin sheaths to compress the axoplasma, which explained the reduction of radial water diffusion after HI (FIG. 10C). DTI was used to evaluate the long-term efficacy of PAI-1 therapy against LPS/HI.

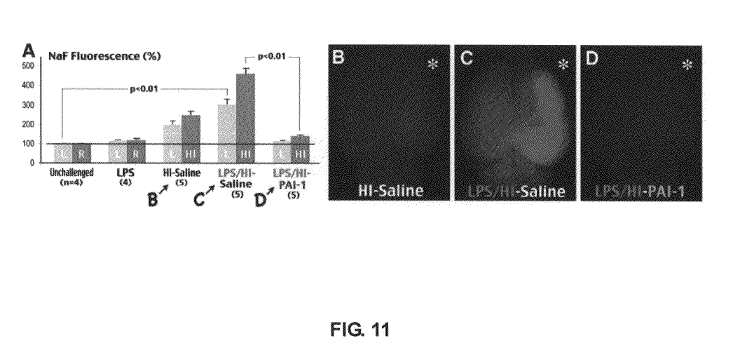

To determine whether ICV-injection of PAI-1 protected against LPS-sensitized HI in rat pups, the effects of PAI-1 on LPS/HI-induced BBB permeability were assessed. IP injection of LPS (0.3 mg/kg) at 4-6 hrs prior to HI in the Vannucci model markedly increased the damage as judged by the BBB-permeability (FIG. 11) and brain tissue loss at 7-day of recovery (data not shown).

The combination of LPS and HI led to increased BBB permeability in rat pups, while PAI-1 administration blocked it (FIG. 11). Quantification of BBB permeability to IP-injected Na=fluorescein (NaF) (n=4,5 each group) is shown in FIG. 11A. FIGS. B-D show images of NaF fluorescence after LPS/HI (asterisk denotes the HI side).

Using NaF as a probe, LPS by itself did not lead to BBB opening, but it increased the baseline as well as the unilateral HI-induced NaF fluorescence in the brain at 15 hrs after insult (asterisks in FIG. 11B, C). ICV injection of PAI-1 (1.9 μg) after LPS/HI reduced the NaF fluorescence to a close-to-baseline level (FIG. 11D).

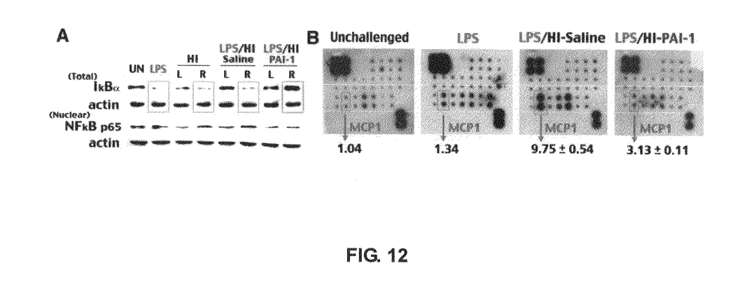

Effects of PAI-1 on NFκB signaling and cytokine production. LPS sensitizes HI brain injury by activating the NFκB signaling pathway and inducing cytokine production. PAI-1 administration as an interceptor of these events was evaluated (FIG. 12). PAI-1 administration dampened LPS/HI-induced NFκB signaling and MCP-1 expression. Immunoblots showed that PAI-1 therapy prevented IκB-degradation and nuclear accumulation of NFκB p65 (FIG. 12B). Cytokine arrays showed reduced MCP=1 expression by PAI-1 (FIG. 12B) (n=3 each for LPS/HI-saline and PAI-1 groups).

LPS, HI, or LPS and HI led to the degradation of IκB protein at 4-8 hrs after insults, but this effect was blocked by post-LPS and HI ICV-injection of PAI-1 (FIG. 12A). PAI-1 administration also resulted in less nuclear accumulation of the NFκB p65 protein at 24 hr after insult. Gel-shift assays examined the effect of PAI-1 on NFκB binding to DNA. An ELISA-based cytokine array to test the effect of PAI-1 therapy showed that LPS strongly induced the production of monocyte chemoattractant protein-1 (MCP-1) in the brain at 24 hr after HI. MCP-1 was significantly suppressed by PAI-1 administration (FIG. 12B, n=3). These results suggest that PAI-1 therapy inhibited the LPS and HI-induced NFκB signaling activation and cytokine production.

Effects of PAI-1 on microglia activation. PAI-1-mediated reduction of MCP-1 expression after HI is important, because MCP-1 has potent monocyte activation and chemotropic effects in the neonatal brain. MCP-1 also plays a critical role in blood-borne cell recruitment after transient focal ischemia in adult brains. Thus, PAI-1 therapy may inhibit microglia activation and/or blood-borne cell recruitment after LPS and HI insult in newborns.

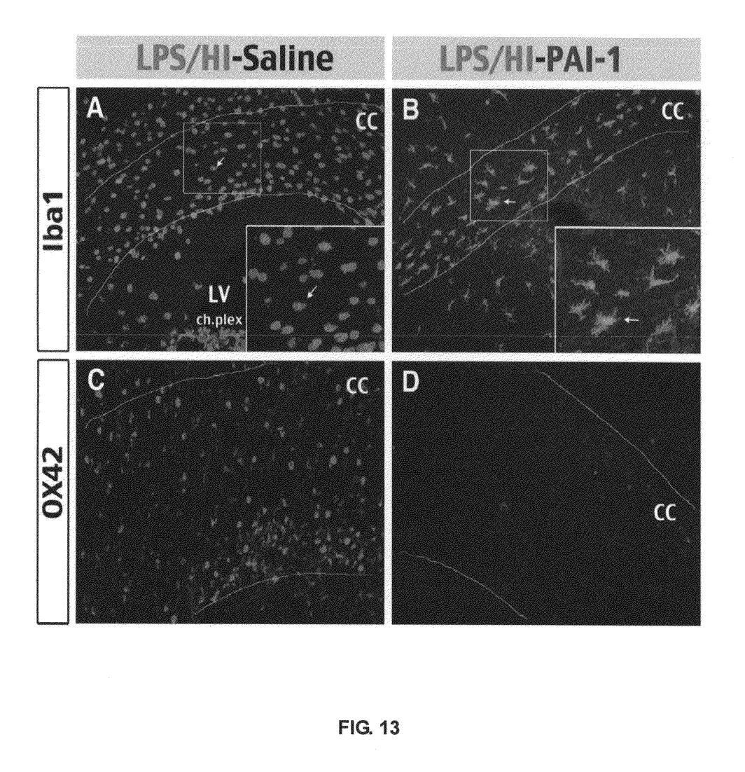

Microglia were stained with Iba1-antibody in the LPS and HI-injured brain. PAI-1 administration greatly decreased the number of Iba1+ cells in the corpus callosum (CC) and cerebral cortex (FIGS. 13A, 13B). The Iba1+ microglia in saline-treated rats adopted an amoeboid-like morphology, while those in PAI-1-treated rats remained in a non-phagocytic state with ramified processes) (inserts in FIGS. 13A, 13B). These data suggested that PAI-1 administration prevented LPS/HI-induced microglia activation.

The number of OX42+ cells were reduced by PAI-1 therapy in LPS and HI-challenged animals (FIGS. 13C, 13D). The OX42 antibody detected both activated microglia and blood-borne macrophages/monocytes. An in-vivo model is expected to show that PAI-1 and/or neruroserpin administration prevents the recruitment of blood-borne monocytes after LPS and HI.

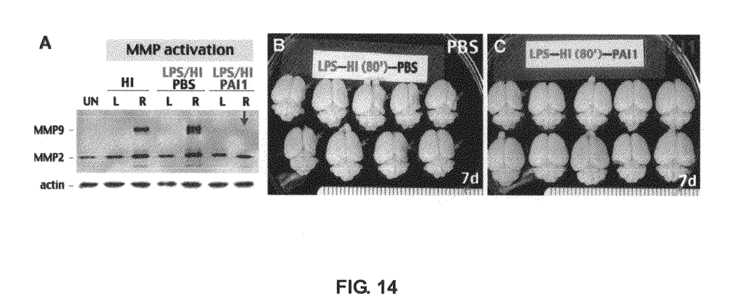

Effects of PAI-1 on MMPs and LPS/HI brain damage are shown in FIG. 14. The effect of PAI-1 against LPS and HI-induced MMP activities was evaluated at 24 hrs (FIG. 14A), and against brain tissue loss was evaluated at seven days of recovery (FIGS. 14B, 14C). PAI-1 administration prevented the LPS and HI-induced MMP9 activity and decreased the amount of brain tissue loss. Administration of PAI-1 prevented LPS/HI-induced MMP activity at 24 hrs (FIG. 14A) and brain tissue loss at seven days of recovery (FIG. 14B).

Effects of PAI-1 on motor development are shown in FIG. 15. One criterion for successful therapy in the neonatal period is whether behavioral-developmental outcome improves beyond short-term histopathological reduction. Body weight and motor functions of LPS and HI-challenged rat pups was assessed up to two months of age, and DTI was performed (FIG. 15). Administration of PAI-1 preserved motor development and WM functions. Saline-injected animals had smaller body weight in the first month after LPS-/HI insult at P7 (FIG. 15A). PAI-1 injected animals had better motor coordination in the rotarod test at P45 (FIG. 15B) (n=9 each). PAI-1 injected animals had better preserved WM integrity than saline-injected animals at 2 two months of age (FIG. 15C) (n=3 for each).

LPS and HI challenge on P7, saline-injected rats initially showed a slower body-weight gain, but gradually caught up after the first month (to 83% of controls, FIG. 15A), but remained significantly impaired in rotarod tests for up to P60 compared with either untouched or PAI-1 injected animals (FIG. 15B, p<0.05 by t-test, n=9). On DTI, saline-injected rats had greatly damaged WM at two months, while PAI-1 administration maintained WM development to a degree indistinguishable from those in untouched rats by inspection (FIG. 15C). Quantitative measurement of several DTI indices (e.g. fractional anisotropy, radial diffusivity, and axial diffusivity) and tractography methods compared WM density. Behavioral and DTI methods test long-term benefits of PAI-1 administration against pre- and postnatal LPS/HI brain injury.

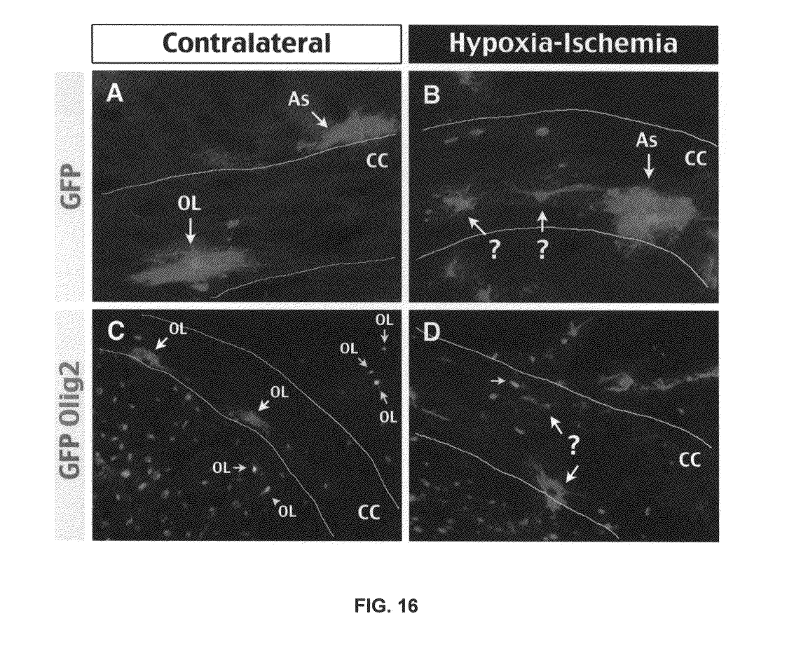

Neonatal HI injury impaired OPC differentiation and motor development in a rat model of perinatal WM injury (FIG. 16). Perinatal infection-HI injury impaired oligodendrocyte development. The surviving/replenishing oligodendrocytes may be stuck in an “arrested maturation” state, based on expression of OL-differentiation markers (e.g. PDGFR, O4, or O1 immunoreactivity), which does not reveal morphological details or ascertain the lineage origin of the aberrant OLs.

Neonatal HI altered the morphology of oligodendrocytes (OL). Nestin-CreER mice were used to visualize OL and astrocyte (As) morphology in normal brain (FIGS. 16A, C). Newborn HI produced aberrant cells (?) with the morphology between OL and As (FIGS. 16B, D).

Tamoxifen-inducible nestin-CreER mice (Burns et al. Glia 57 (2009) 1115) were crossed to a stop-floxed EGFP-reporter reporter line, and injected with tamoxifen at E18 to label the perinatal SVZ glioblast progenitors. The mice were stressed by HI at P10, received BrdU from P11 to P13, and killed at P30 to examine the morphology of OLs and astrocytes (As). On the contralateral side, there were many mature OLs extending multiple processes parallel to the trajectory of corpus callosum (CC) and the majority of astrocytes were located outside the CC (FIGS. 16A, 16C). In contrast, on the HI-challenged side, many aberrant-shaped cells (indicated by “?” in FIGS. 16B, 16D) with the morphology between OL and astrocyte were found in the CC. Many of these aberrant cell contained BrdU (data not shown) indicating they were born after P10, which is unusual for astrocytes but typical for the birthdates of OLs. Together, these results suggested that perinatal HI (or LPS) injury stimulates astrocytic differentiation by oligodendrocyte progenitors (OPCs). There may be an ectopic migration by astrocytes. Using a genetic fate-mapping strategy and live-imaginf will distinguish between the two scenarios.

Cerebral palsy (CP) is frequently a consequence of perinatal brain injury such as hypoxic-ischemic encephalopathy (HIE) and germinal matrix hemorrhage (GMH). However, not all infants suffering from HIE or GMH will develop CP at two years of age. There is no laboratory test in the neonatal period to predict which infants are at a greater risk of contracting CP. This creates high anxiety in parents of high-risk infants, but also underscores the inability to stop the CP-causing brain destruction process at the neonatal stage. Development of CP predictive tests will improve the quality of neonatal care, advance understanding of the etiology of CP, and lead to more effective therapy.

Using animal model mechanistic and therapeutic studies, tPA and other related plasminogen activators in the newborn brain tissue have been identified as mediators of injury leading to HIE and CP. While tPA is dissolves blood clots and is the only FDA-approved agent for treating acute ischemic stroke, tPA outside of blood vessels has deleterious effects in the brain. Extravascular tPA plays a role in causing severe HI brain injury in newborn animals. This same “ectopic tPA” mechanism, if applicable to human infants, will lead to a prognostic test of CP and an effective brain protection strategy for HIE.

tPA protein levels in the cerebrospinal fluid (CSF) of 40 infants with HIE/GMH and 20 non-HIE/GMH infants are examined to gauge tPA activity in the infant brain. These high-risk infants will be monitored at a special clinic until two years of age to determine whether high levels of CSF-tPA in the neonatal stage is an ominous sign of severe brain damage and CP. Brain autopsy samples from twelve infants who died of HIE/GMH-related or non-related causes are evaluated to compare the tPA expression levels in brain tissue. The method combines two biochemical and pathological methods. The following references are expressly incorporated by reference in their entirety:

Adhami F, Yu D, Yin W, Schloemer A, Burns K A, Liao G, Degen J L, Chen J, Kuan C Y (2008) Deleterious effects of plasminogen activators in neonatal cerebral hypoxia-ischemia. Am J Pathol 172:1704-1716.
Asahi M, Wang X, Mori T, Sumii T, Jung J C, Moskowitz M A, Fini M E, Lo EH (2001) Effects of matrix metalloproteinase-9 gene knock-out on the proteolysis of blood-brain barrier and white matter components after cerebral ischemia. J Neurosci 21:7724-7732.
Berkenpas M B, Lawrence D A, Ginsburg D (1995) Molecular evolution of plasminogen activator inhibitor-1 functional stability. Embo J 14:2969-2977.
Chahboune H, Ment L R, Stewart W B, Ma X, Rothman D L, Hyder F (2007) Neurodevelopment of C57B/L6 mouse brain assessed by in vivo diffusion tensor imaging. NMR Biomed 20:375-382.
Cinelli P, Madani R, Tsuzuki N, Vallet P, Arras M, Zhao C N, Osterwalder T, Rulicke T, Sonderegger P (2001) Neuroserpin, a neuroprotective factor in focal ischemic stroke. Mol Cell Neurosci 18:443-457.
del Zoppo G J, Milner R, Mabuchi T, Hung S, Wang X, Berg GI, Koziol J A (2007) Microglial activation and matrix protease generation during focal cerebral ischemia. Stroke 38:646-651.
Derrick M, Luo N L, Bregman J C, Jilling T, J i X, Fisher K, Gladson C L, Beardsley D J, Murdoch G, Back SA, Tan S (2004) Preterm fetal hypoxia-ischemia causes hypertonia and motor deficits in the neonatal rabbit: a model for human cerebral palsy? J Neurosci 24:24-34.
Ferriero D M (2004) Neonatal brain injury. N Engl J Med 351:1985-1995.
Gu Z, Cui J, Brown S, Fridman R, Mobashery S, Strongin A Y, Lipton S A. (2005) A highly specific inhibitor of matrix metalloproteinase-9 rescues laminin from proteolysis and neurons from apoptosis in transient focal cerebral ischemia. J Neurosci. 25:6401-8.
Heo J H, Lucero J, Abumiya T, Koziol J A, Copeland B R, del Zoppo G J (1999) Matrix metalloproteinases increase very early during experimental focal cerebral ischemia. J Cereb Blood Flow Metab 19:624-633. ladecola C (2004) Neurovascular regulation in the normal brain and in Alzheimer's disease. Nat Rev Neurosci 5:347-360.
Lo E H, Broderick J P, Moskowitz M A (2004) tPA and proteolysis in the neurovascular unit. Stroke 35:354-356.
Moseley M E, Cohen Y, Mintorovitch J, Chileuitt L, Shimizu H, Kucharczyk J, Wendland M F, Weinstein P R (1990) Early detection of regional cerebral ischemia in cats: comparison of diffusion- and T2-weighted MRI and spectroscopy. Magn Reson Med 14:330-346.
McColl B W, Rothwell N J, Allan S M (2008) Systemic inflammation alters the kinetics of cerebrovascular tight junction disruption after experimental stroke in mice. J Neurosci 28:9451-9462.
Mun-Bryce S, Rosenberg G A (1998) Matrix metalloproteinases in cerebrovascular disease. J Cereb Blood Flow Metab 18:1163-1172.
Nagai N, De Mol M, Lijnen H R, Carmeliet P, Collen D (1999) Role of plasminogen system components in focal cerebral ischemic infarction: a gene targeting and gene transfer study in mice. Circulation 99:2440-2444.
Rice J E 3rd, Vannucci R C, Brierley J B. (1981) The influence of immaturity on hypoxic-ischemic brain damage in the rat. Ann Neurol. 19:131-41.
Sappino A P, Madani R, Huarte J, Belin D, Kiss J Z, Wohlwend A, Vassalli J D. (1993) Extracellular proteolysis in the adult murine brain. J Clin Invest. 92:679-85.
Siao C J, Tsirka S E (2002) Tissue plasminogen activator mediates microglial activation via its finger domain through annexin II. J Neurosci 22:3352-3358.
Su E J, Fredriksson L, Geyer M, Folestad E, Cale J, Andrae J, Gao Y, Pietras K, Mann K, Yepes M, Strickland D K, Betsholtz C, Eriksson U, Lawrence D A (2008) Activation of PDGF-CC by tissue plasminogen activator impairs blood-brain barrier integrity during ischemic stroke. Nat Med 14:731-737.
Sumii T, Lo E H. (2002) Involvement of matrix metalloproteinase in thrombolysis-associated hemorrhagic transformation after embolic focal ischemia in rats. Stroke. 33:831-6.
Svedin P, Hagberg H, Savman K, Zhu C, Mallard C (2007) Matrix metalloproteinase-9 gene knock-out protects the immature brain after cerebral hypoxia-ischemia. J Neurosci 27:1511-1518.
Tsirka S E, Gualandris A, Amaral D G, Strickland S. (1995) Excitotoxin-induced neuronal degeneration and seizure are mediated by tissue plasminogen activator. Nature. 377:340-4.
Volpe J J. (2008) “Neurology of the newborn”. 5/e, Saunders Elsevier, Philadelphia, Pa.
Wang X, Lee S R, Arai K, Lee S R, Tsuji K, Rebeck G W, Lo E H (2003) Lipoprotein receptor-mediated induction of matrix metalloproteinase by tissue plasminogen activator. Nat Med 9:1313-1317.
Wang Y F, Tsirka S E, Strickland S, Stieg P E, Soriano S G, Lipton S A (1998) Tissue plasminogen activator (tPA) increases neuronal damage after focal cerebral ischemia in wild-type and tPA-deficient mice. Nat Med 4:228-231.
Yepes M, Sandkvist M, Wong M K, Coleman T A, Smith E, Cohan S L, Lawrence D A (2000) Neuroserpin reduces cerebral infarct volume and protects neurons from ischemia-induced apoptosis. Blood 96:569-576.

Other variations or embodiments will also be apparent to one of ordinary skill in the art from the description. Thus, the foregoing embodiments are not to be construed as limiting the scope of the following claims.

What is claimed is:

1. A method of preventing or ameliorating cerebral palsy, the method comprising administering plasminogen activator inhibitor-1 (PAI-1) in an effective regimen to a newborn to decrease the effects of at least one of a hypoxia ischemia (HI) injury selected from hypoxia ischemia encephalopathy (HIE), periventricular malacia (PVL), or germinal matrix hemorrhage with ventricular dilation (PHVD) in the newborn, thereby preventing or ameliorating cerebral palsy.

2. A method of ameliorating hypoxia ischemia (HI) encephalopathy-related diseases resulting from a hypoxia ischemia (HI) injury, the method comprising administering plasminogen activator inhibitor-1 (PAI-1) in an effective regimen to a newborn to result in at least one of a decrease of HI-induced matrix metalloproteinase 9 (MMP-9) activity, a decrease of HI-induced blood brain barrier permeability and/or brain edema, or a decrease of HI-induced white-matter damage and/or cortical degeneration, relative to a control individual, thereby ameliorating HI encephalopathy-related diseases resulting from an HI injury.

3. The method of claim 1 or claim 2 wherein the HI injury is accompanied by infection.

4. The method of claim 3 wherein infection occurs before, during, or after HI injury.

5. The method of claim 1 or claim 2 wherein PAI-1 administration results in decreased tissue-type plasminogen activator (tPA) and/or urokinase-type plasminogen activator (uPA) effect.

6. The method of claim 1 or claim 2 wherein administering PAI-1 results in increased oligodendrocyte progenitor survival and differentiation, decreased hypomyelination, decreased monocyte brain infiltration, decreases NF-κB activation, decreased cytokine production, and/or decreased microglia activation relative to a control individual.

7. The method of claim 3 wherein the infection is intrauterine or after birth.

8. The method of claim 1 or claim 2, further comprising monitoring the effect of PAI-1 administration using MRI-diffusion tensor imaging and comparing white matter development in a patient administered PAI-1 with white matter development of a patient not administered PAI-1.

9. A method of diagnosis of hypoxia ischemia encephalopathy-related diseases in an individual by measurement of members of the plasminogen activator signaling pathways in the individual relative to a control individual, and diagnosing a hypoxia ischemia encephalopathy-related disease in the individual if at least one of plasmin, tissue-type plasminogen activator (tPA), or urokinase-type plasminogen activator (uPA) is elevated relative to a control individual.

10. The method of claim 9 wherein a protein concentration or activity of at least one of plasmin, tPA, or uPA is elevated relative to a control individual.
